(19) United States
(12) Patent Application Publication
(10) Pub. No.: US 2025/0325735 A1
(43) Pub. Date: Oct. 23, 2025

(54) CONFIGURATION OF A DIALYSIS MACHINE FOR EXTRACORPOREAL BLOOD THERAPY

(71) Applicant: GAMBRO LUNDIA AB, Lund (SE)

(72) Inventors: Gunilla ANDERSSON, Harlösa (SE); Anders FELDING, Limhamn (SE)

(21) Appl. No.: 18/864,090

(22) PCT Filed: May 10, 2023
(86) PCT No.: PCT/EP2023/062347

§ 371 Date: Nov. 8, 2024

(30) Foreign Application Priority Data
May 12, 2022 (SE) 2250572-1

(51) Int. Cl. A61M 1/16; A61M 60/113

(52) U.S. Cl. CPC A61M 1/1603; A61M 60/113; A61M 2205/3334; A61M 2205/502; A61M 2205/52

(57) ABSTRACT

A dialysis machine for extracorporeal blood therapy is configured to interface with a disposable blood line that is available in at least two different sizes. A memory in the dialysis machine is arranged to store configuration data that associates the different sizes of the blood line with a respective predefined limit value of fluid removal when the dialysis machine is operated by a controller to perform extracorporeal blood therapy. The controller is configured to execute a configuration procedure, which comprises obtaining size data indicative of a selected size of the blood line for use in the extracorporeal blood therapy in relation to a patient; determining, by use of the configuration data and based on the selected size, a maximum rate of said fluid removal from blood, and configuring the dialysis machine to maintain the fluid removal below the maximum rate during the extracorporeal blood therapy.

### TECHNICAL FIELD

The present disclosure relates generally to dialysis machines for extracorporeal blood therapy, and in particular to a technique of configuring such a dialysis machine.

### BACKGROUND ART

Extracorporeal blood therapy is a renal replacement therapy (RRT) that is performed to replace the normal blood-filtering function of the kidneys. In extracorporeal blood therapy, blood is extracted from a patient, treated in a blood filtration unit and returned to the patient. The blood filtration unit is known as a blood filter or “dialyzer”, and defines first and second chambers separated by a semi-permeable membrane. While blood is pumped through the first chamber, fluid and waste products are removed from the blood by transfer through the semi-permeable membrane into the second chamber. Depending on modality, a dialysis fluid may or may not be pumped through the second chamber. Different modalities of extracorporeal blood therapy include hemofiltration (HF), hemodialysis (HD) and hemodiafiltration (HDF).

Extracorporeal blood therapy is performed by a dialysis machine, which is configured to expose mechanical interfaces to various pumps, fluid connectors, sensors, etc. Before treatment, a disposable set is mounted to the dialysis machine. The disposable set comprises the dialyzer and tubing or fluid lines that define fluid channels in relation to the dialyzer. After treatment, the disposable set is discarded.

Dialysis machines are costly devices that are manufactured in relatively small quantities and have long operational lifetime. The small quantities give little room for diversification and dialysis machines are typically configured with an ability to execute many different modalities of extracorporeal blood therapy. Dialysis machines are also used irrespective of patient characteristics. The adaptation of a dialysis machine to different modalities and patient characteristics is made through the use of different disposable sets and by a caretaker calculating and entering operating parameters for the dialysis machine to meet the needs of the specific patient.

For example, pediatric dialysis may be performed by use of generic dialysis machines. Since the total blood volume is much smaller in children compared to adults, the disposable set is adapted to contain a smaller amount of blood, typically by having a smaller inner diameter of fluid channels. However, when generic dialysis machines are used for pediatric dialysis, there is a risk that the caretaker configures the dialysis machine by entering operating parameters that would be acceptable for an adult patient but that are unsuitable or even harmful to a child. The dialysis machine, being adapted for extracorporeal blood therapy of adults, may not be capable of detecting such an unsuitable configuration.

### SUMMARY

It is an objective to at least partly overcome one or more limitations of the prior art.

One objective is to provide a technique that reduces the risk of dialysis machines being incorrectly configured when used for patients with small total blood volume.

Another objective is to provide such a technique that is simply retrofitted onto existing dialysis machines.

One or more of these objectives, as well as further objectives that may appear from the description below, are at least partly achieved by a dialysis machine, a computer-implemented method, and a computer-readable medium, embodiments thereof being defined by the dependent claims.

A first aspect is a dialysis machine that comprises a first pumping arrangement, which is configured to interface with a disposable blood line in fluid communication with a first compartment of a dialyzer, the blood line being available in at least two different sizes. The dialysis machine further comprises a second pumping arrangement which is configured to interface with a line set in fluid communication with a second compartment of the dialyzer, the second compartment being separated from the first compartment by a semipermeable membrane. The second pumping arrangement is operable to control fluid removal from blood in the first compartment through the semipermeable membrane. The dialysis machine further comprises a user interface for interaction with a user of the dialysis machine, a memory for storing configuration data that associates the different sizes of the blood line with a respective predefined limit value of said fluid removal, and a controller connected to the memory and the user interface and configured to operate the first and second pumping arrangements to perform extracorporeal blood therapy. The controller is further configured, during a configuration procedure, to: obtain size data indicative of a selected size of the blood line for use in the extracorporeal blood therapy in relation to a patient; determine, by use of the configuration data and based on the selected size, a maximum rate of said fluid removal from blood; and configure the dialysis machine to maintain the fluid removal below the maximum rate during the extracorporeal blood therapy.

In some embodiments, the controller is configured to obtain the size data through the user interface.

In some embodiments, the configuration data comprises at least one predefined limit value that is specific to pediatric dialysis and at least one predefined limit value that is specific to non-pediatric dialysis.

In some embodiments, the controller is further configured to obtain a current weight of the patient, and the controller is configured to determine the maximum rate based on the current weight of the patient and the predefined limit value that is associated with the selected size.

In some embodiments, the predefined limit value defines a maximum rate of fluid removal per unit weight.

In some embodiments, the controller is configured to obtain the current weight by prompting a user to enter the current weight through the user interface.

In some embodiments, the controller is configured to obtain the current weight only if the selected size falls within a specific size range.

In some embodiments, the specific size range represents blood lines used for pediatric dialysis.

In some embodiments, the controller comprises a first module configured to perform the configuration procedure, and a second module configured to estimate treatment efficiency of the extracorporeal blood therapy based on conductivity data from a sensor arrangement in the dialysis machine, the second module being operative to allow input of the current weight through the user interface, and the first module is configured to obtain the current weight by use of the second module.

In some embodiments, the configuration data further associates the different sizes with a respective weight range, and the controller is further configured to evaluate the current weight in relation the weight range associated with the selected size of the blood line and, if the current weight deviates from the weight range, present a weight alert on the user interface.

In some embodiments, the controller is further configured to present the maximum rate on the user interface.

In some embodiments, the controller is further configured to determine, based on input data obtained through the user interface, a selected rate of fluid removal for use in the extracorporeal blood therapy, evaluate the selected rate in relation to the maximum rate, and, if the selected rate exceeds the maximum rate, present a rate alert on the user interface.

In some embodiments, the first pumping arrangement comprises a peristaltic pump, and the different sizes correspond to different inner diameters of a portion of the blood line configured for engagement with one or more rollers of the peristaltic pump.

A second aspect is a computer-implemented method of configuring a dialysis machine to perform extracorporeal blood therapy. The dialysis machine comprises a user interface; a first pumping arrangement, which is configured to interface with a disposable blood line in fluid communication with a first compartment of a dialyzer, the blood line being available in at least two different sizes; and a second pumping arrangement which is configured to interface with a line set in fluid communication with a second compartment of the dialyzer, the second compartment being separated from the first compartment by a semipermeable membrane, and the second pumping arrangement being operable to control fluid removal from blood in the first compartment through the semipermeable membrane during the extracorporeal blood therapy. The computer-implemented method comprises: obtaining size data indicative of a selected size of the blood line for use in the extracorporeal blood therapy in relation to a patient; retrieving, from a memory, configuration data that associates the different sizes of the blood line with a respective predefined limit value of said fluid removal; determining, by use of the configuration data and based on the selected size, a maximum rate of said fluid removal from blood; and configuring the dialysis machine to maintain the fluid removal below the maximum rate during the extracorporeal blood therapy.

Any embodiment of the first aspect, as found herein, may be adapted and implemented as an embodiment of the second aspect.

A third aspect is a computer-readable medium comprising computer instructions which, when executed by a processor, cause the processor to perform the method of the second aspect or any of its embodiments.

The foregoing aspects provide a technique of supporting a user while configuring a dialysis machine for extracorporeal blood therapy, to mitigate the risk that the dialysis machine is incorrectly configured when used for a patient with small total blood volume, for example a child. The configuration procedure is based on the insight that the maximum rate of fluid removal may be determined based on the size of the blood line to be used in therapy, since this size needs to be known for the dialysis machine to operate the first pumping arrangement to provide a correct blood flow rate through the dialyzer. Thus, by the configuration procedure, the dialysis machine is inherently configured to maintain the fluid removal below a maximum rate that is adapted to the total blood volume of the patient. Further, the provision of the configuration data enables simple updating of the predefined limit values, for example in accordance with the needs of a specific clinic, ward or dialysis machine. The configuration data may also be simply updated whenever a new size of blood line is available for installation in the first pumping arrangement. Still further, the configuration procedure is simple to implement on pre-existing dialysis machines, for example by updating a control program stored in the controller and by providing appropriate configuration data.

Still other objectives, aspects, embodiments and technical effects, as well as features and advantages may appear from the following detailed description, from the attached claims as well as from the drawings.

### BRIEF DESCRIPTION OF THE DRAWINGS

FIG. 1A is a front view of an example dialysis machine for extracorporeal blood therapy, FIG. 1B is a diagram of the dialysis machine in FIG. 1A when configured and operated for extracorporeal blood therapy, and FIG. 1C is a block diagram of an example controller for the dialysis machine in FIG. 1A.

FIG. 2A is a front view of a blood pump in the dialysis machine of FIG. 1A, and FIGS. 2B-2C are side views of pump segments of different sizes for installation in the peristaltic pump of FIG. 2A.

FIG. 3A is a block diagram of an example dialysis machine, and FIG. 3B depicts configuration data for use in configuring the dialysis machine.

FIGS. 4A-4B are flow charts of example computer-implemented methods of configuring a dialysis machine.

FIGS. 5A-5E show example messages displayed on a user interface by the methods in FIGS. 4A-4B.

FIG. 6 is a block diagram of a controller comprising a separate software module for monitoring treatment efficiency.

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more”, even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments.

As used herein, the terms “multiple”, “plural” and “plurality” are intended to imply provision of two or more elements. The term “and/or” includes any and all combinations of one or more of the associated listed elements.

It will furthermore be understood that although the terms first, second, etc. may be used herein to describe various elements, these elements should not be limited by these terms. These terms are only used to distinguish one element from another. For example, a first element could be termed a second element, and, similarly, a second element could be termed a first element, without departing the scope of the present disclosure.

Well-known functions or constructions may not be described in detail for brevity and/or clarity. Unless otherwise defined, all terms (including technical and scientific terms) used herein have the same meaning as commonly understood by one of ordinary skill in the art to which this disclosure belongs.

Like reference signs refer to like elements throughout.

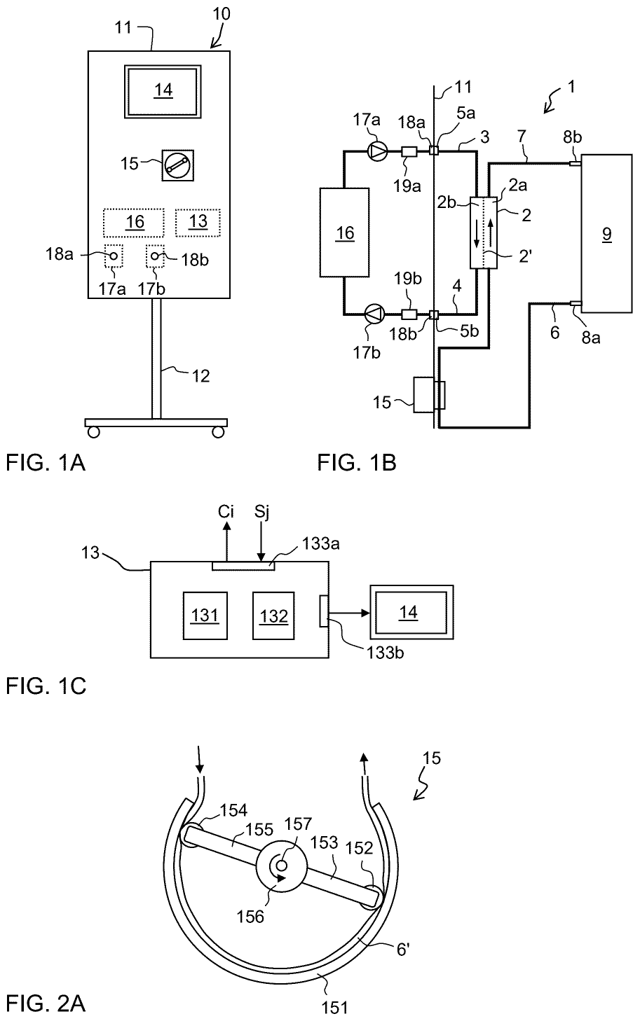

FIG. 1A is a front view of an example dialysis machine 10, which will be used for describing various embodiments. The dialysis machine 10, also known as “monitor” in the art, is configured to be combined with a set of disposable components (“disposable set”) to define a dialysis system, which is operable to perform extracorporeal blood therapy. The dialysis machine 10 comprises a housing or chassis 11, which encloses various internal components (indicated by dashed lines) and defines mechanical interfaces for engagement with the disposable set, in accordance with conventional practice. In the illustrated example, the housing 11 is mounted on a stand 12 on wheels and includes a control device 13, a user interface (UI) device 14, a blood pump 15, a fluid supply arrangement 16, treatment fluid pumps 17a, 17b and fluid ports 18a, 18b. The control device 13 is configured to control the operation of the dialysis machine 10. The UI device 14 is electrically connected to control device 13. The term “UI device” is intended to include any and all devices that are capable of performing guided human-machine interaction comprising presentation of information and receipt of input. Thus, the UI device 14 may comprise a combination of a presentation device and data entry hardware. The presentation device may comprise one or more of a display, speaker, projector, lamps, etc. The data entry hardware may include one or more of a keyboard, keypad, computer mouse, control buttons, touch panel, microphone and voice control functionality, camera and gesture control functionality, etc. In one implementation, the UI device 14 is or comprises a touch-sensitive display, also known as touch screen.

The blood pump 15 is configured for engagement with a tubing or fluid line in the disposable set and is operable to drive blood through the tubing. In the illustrated example, the blood pump 15 is a peristaltic pump. Although a single blood pump 15 is shown in FIG. 1A, it is conceivable that the dialysis machine 10 comprises plural blood pumps. Thus, generally, dialysis machine 10 comprises a first pumping arrangement, which is operable to pump blood through one or more fluid lines of the disposable set. The fluid supply arrangement 16 is configured to provide treatment fluid for use in the extracorporeal blood therapy. The fluid supply arrangement 16 may be configured to provide the treatment fluid from pre-filled bags, which are installed on the dialysis machine 10, or generate the treatment fluid on demand, for example by mixing one or more concentrates with purified water. The treatment fluid pump (“supply pump”) 17a is operable to supply fresh treatment fluid to the fluid port 18a, and the treatment fluid pump (“effluent pump”) 17b is operable to receive spent treatment fluid at the fluid port 18a. The pumps 17a, 17b may be seen to form (part of) a second pumping arrangement of the dialysis machine 10. As used herein, “spent” treatment fluid refers to treatment fluid that has been used for the extracorporeal blood treatment and contains fluid and solutes extracted from blood.

FIG. 1B is a side view of the dialysis machine 10 when configured for hemodialysis (HD). Internal components are shown to the left of the casing 11, and disposable components that are attached to mechanical interfaces on the casing 11 are shown to the right. The blood pump 15 is arranged to project from the casing 11 to expose a mounting portion for engagement with a blood line 6 (below). The supply pump 17a is arranged in an output fluid path that extends from the fluid supply arrangement 16 to the fluid port 18a, and the effluent pump 17b is arranged in an input fluid path that extends from the fluid port 18b to the fluid supply arrangement 16. The fluid supply arrangement 16 may be configured to regenerate the spent treatment fluid into fresh treatment fluid, or direct the spent treatment fluid to a drain (not shown) for disposal.

In the illustrated example of FIG. 1B, a respective sensor 19a, 19b is arranged on the output fluid path and the input fluid path to measure a property of the passing treatment fluid. The measured property is a concentration-related parameter, such as electrical conductivity or a concentration of one or more substances. Output signals of the sensors 19a, 19b, generated during a specific test sequence performed by the dialysis machine 10, may be processed by the control device 13 for determination of the efficiency of on-going blood therapy, as is well-known in the art. The use of the sensors 19a, 19b will be further described below with reference to FIG. 6.

The disposable components, as shown to the right of the casing 11 in FIG. 1B, comprise a blood filter 2, commonly known as a “dialyzer”. The dialyzer 2 comprises a semi-permeable membrane 2′, which separates the dialyzer 2 into a first compartment 2a for blood and a second compartment 2b for treatment fluid. The disposable components further comprise a set of fluid lines (“line set”). The line set includes a supply line 3 for fresh treatment fluid, and a drain line 4 for spent treatment fluid. The supply line 3 comprises a connector 5a for releasable connection to the port 18a, and the drain line 4 comprises a connector 5b for releasable connection to the port 18b. By connection to the ports 18a, 18b, the fluid lines 3, 4 are “interfaced” with the fluid pumps 17a, 17b. The fluid lines 3, 4 and are releasably or permanently connected to the dialyzer 2 in fluid communication with compartment 2b. The line set further comprises a blood in-flow line 6 and a blood out-flow line 7, which are releasably or permanently connected to the dialyzer 2 in fluid communication with compartment 2a. Access devices 8a, 8b are arranged on the distal ends of the fluid lines 6, 7 and configured for connection to block 9, which may represent a patient or a blood reservoir. When connected to a patient, the respective access device 8a, 8b may be a needle or a catheter, as is well-known in the art. As shown, the blood in-flow line 6 is arranged in engagement with the blood pump 15. Instead of a collection of tubes, as shown, the line set may be implemented as an integrated cassette, as in well-known in the art.

In a variant, not shown, the fluid pumps 17a, 17b are instead peristaltic pumps, which are arranged to project from the casing 11 for engagement with the supply line 3 and the drain line, respectively. In such a variant, the supply line 3 may extend from the fluid supply arrangement 16, for example a pre-filled bag of treatment fluid, to the dialyzer 2, and the drain line 4 may extend from the dialyzer 2 to drain.

FIG. 1C is schematic view of the control device (“controller”) 13 according to an embodiment. The control device 13 is configured to receive measurement signals, collectively designated by Sj, and generate control signals, collectively designated by Ci. The control device 13 may be configured to generate the control signals Ci in accordance with a control program (software) and based on the measurement signals Sj. The control signals Ci are provided to various functional components of the dialysis machine 10, including but not limited to the blood pump 15 and the fluid pumps 17a, 17b. The measurement signals Sj are generated by a variety of sensors (not shown) in the dialysis machine 10 and/or in the disposable set, such as pressure sensors, flow meters, temperature sensors, blood leak sensors, air detectors, etc. The measurement signals Sj also include signals from the sensors 19a, 19b, if present. The control device 13 comprises circuitry that includes one or more processors 131 and computer memory 132. The control program is stored in the memory 132 and executed by the processor(s) 131. The control program may be supplied to the control device 13 on a computer-readable medium, which may be a tangible (non-transitory) product (e.g., magnetic medium, optical disk, read-only memory, flash memory, etc.) or a propagating signal. In the illustrated example, the control device 13 comprises a first signal interface 133a for providing control signals Ci and receiving measurement signals Sj, and a second signal interface 133b for connection to the UI device 14. The first and second interfaces 133a, 133b may be combined into a single interface.

When the disposable set has been connected to the dialysis machine 10, as shown in FIG. 1B, the control device 13 is operable to control the dialysis machine 10 to perform the extracorporeal blood therapy. During such therapy, the blood pump 15 is operated to pump blood from the patient 9, through the compartment 2a, and back to the patient, and the fluid pumps 17a, 17b are operated to pump treatment fluid through compartment 2b, as indicated by arrows. Thereby, fluid and solutes are exchanged between the blood and the treatment fluid through the semi-permeable membrane 2′. Extraction of fluid from blood to the treatment fluid is controlled by the fluid pumps 17a, 17b and is known as ultrafiltration (UF). Exchange of solutes is mainly driven by diffusion. In HD, the treatment fluid that is pumped through the dialyzer 2 is commonly denoted “dialysis fluid”.

FIGS. 1A-1C are simplified and merely given to provide context for the following description. Thus, the dialysis machine 10 and the disposable set may include further components not shown or discussed herein, including additional fluid pumps to enable other modalities of extracorporeal blood therapy. While the configuration of the dialysis machine 10 and the disposable set in FIGS. 1A-1B has been given for hemodialysis (HD), the present disclosure is equally applicable to other modalities such as hemofiltration (HF) and hemodiafiltration (HDF). In HF, treatment fluid is not supplied to the dialyzer 2, and UF is controlled by the effluent pump 17b, which is operated to draw fluid through the membrane 2′ by convention. The extracted fluid is replaced in part or completely with treatment fluid (“replacement fluid”), which is infused into blood line 6 or 7. HDF combines HD and HF and thus involves pumping fresh dialysis fluid through the compartment 2b of the dialyzer 2 in accordance with HD and replacing the extracted fluid in part or completely with replacement fluid in accordance with HF. It is realized that one and the same dialysis machine 10 may be operated to perform different modalities by installation of a dedicated disposable set and entry of dedicated control settings for the control program in the dialysis machine 10.

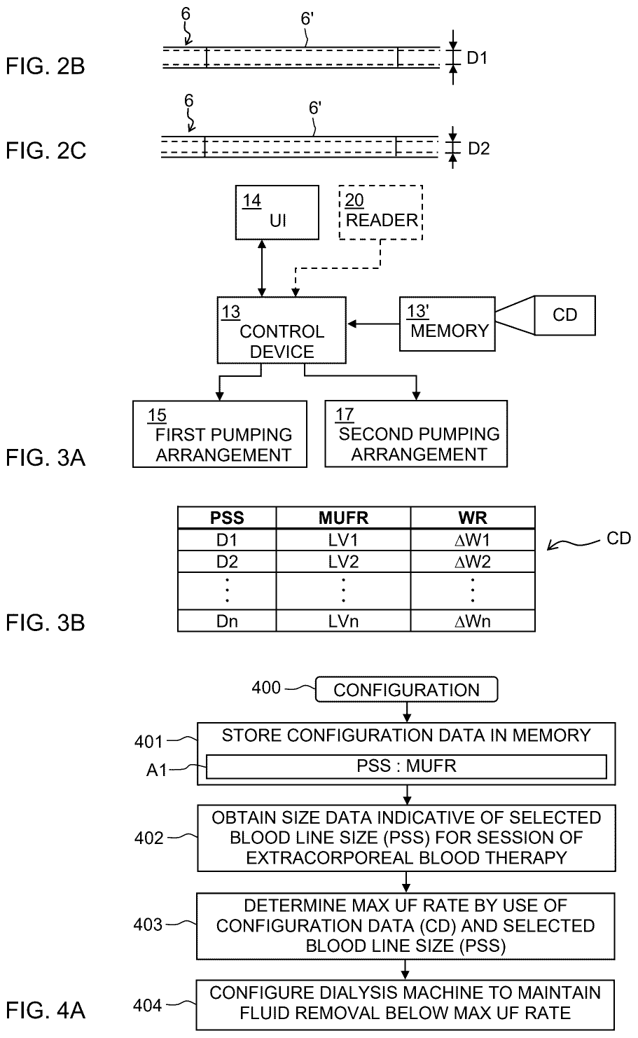

FIG. 2A is front view of an example pump head of the blood pump 15 on the dialysis machine 10. The blood pump 15 is a peristaltic pump of rotary type and the pump head comprises a fixed frame 151, which defines a curved support surface for a line segment 6′ of the blood line 6, and a concentrically arranged rotor. The line segment 6′ is also known as “pump segment” in the art. The pump segment 6′ is installed to extend in parallel along the curved support surface. The pump segment 6′ is a flexible tubing portion. In some implementations, the pump segment 6′ may differ in structure from other parts of the blood line 6. For example, the pump segment 6′ may be reinforced and/or made of thicker and/or more sturdy material to sustain the engagement forces from the pump 15 over time. In the illustrated example, the rotor comprises two rollers 152, 154 which are rotatably arranged on a respective arm 153, 155 which is fixedly arranged on a central hub 156. The rollers 152, 154 are also known as shoes, wipers or lobes. A drive shaft 157 is fixedly attached to the hub 156 and connected for rotation by an electric motor (not shown). As the rotor turns, part of the pump segment 6′ is compressed by the respective roller 152, 154 and thereby pinched closed (“occluded”) so that fluid is driven along the pump segment 6′. The rotor of the pump 15 may carry more than two rollers 152, 154.

As understood from the foregoing, the dialysis machine 10 comprises a first pumping arrangement, which includes the blood pump 15, and a second pumping arrangement, which includes at least the effluent pump 17b. The first pumping arrangement is configured to interface with a disposable blood line 6 in fluid communication with compartment 2a of the dialyzer 2. The second pumping arrangement is configured to interface with a line set 3, 4 in fluid communication with compartment 2b of the dialyzer 10 and is operable to control fluid removal from blood in the first compartment through the membrane 2′.

As noted in the Background section, the caretaker may have pump segments 6′ of different sizes to choose from when installing the disposable set on the dialysis machine 10. The pump segment 6′ may be integrated with the blood line 6 or separately provided for attachment to connecting portions of the blood line 6. In this context, the size of the pump segment 6′ refers to the amount of blood that is pumped per unit time by the blood pump 15 when it engages the pump segment 6′. Typically, the size of the pumping segment 6′ is given by its inner diameter. FIGS. 2B-2C illustrate pump segments 6′ with different inner diameters D1, D2. In a non-limiting example, pump segments may be available with inner diameters of 8 mm, 6.4 mm and 4 mm. The rest of the blood line 6 may or may not have the same inner diameter as the pump segment 6′. As used herein, any reference to the size of a blood line 6 refers to the size of its pump segment 6′. The size of the pump segment is abbreviated PSS in the following.

FIG. 3A is a block diagram of an example dialysis machine, for example of the type shown in FIG. 1A. A control device 13 is configured to control the operation of the dialysis machine, by providing control signals to a first pumping arrangement 15, to thereby operate the blood pump, and a second pumping arrangement 17, to thereby operate at least the effluent pump 17b and possibly the supply pump 17a (depending on modality). The control device 13 is further connected to the UI device 14, through which the control device 13 is operable to receive control settings and other input data from a user of the dialysis machine 10 and provide instructions to the user. The control settings define the operation of the dialysis machine during a therapy session. Examples of control settings include the total amount of fluid removal for a therapy session (total UF), duration of the therapy session (treatment time, TT), flow rate of blood into dialyzer (BFR), flow rate of treatment fluid into the dialyzer (DFR), etc.

The control device 13 is further connected to a memory 13′, which may be part of the computer memory 132 (FIG. 1C) or an external memory unit. The memory 13′ is operable to store configuration data, CD, which associates different pump segment sizes (PSS) with a respective predefined limit value of the fluid removal to be achieved by the dialysis machine 10 during on-going therapy. The use and structure of the configuration data will be described further below. As indicated by dashed lines, the control device 13 may also be connected to a reader 20, which is operable to detect an identifying code of a pump segment before, during or after its installation on the dialysis machine 10. The identifying code may be provided on an external packaging for the pump segment or on the pump segment itself. For example, the reader 20 may be an optical scanner or an RFID reader, and the identifying code may be provided in a corresponding format.

The technical solution described herein aims at mitigating the risk that the dialysis machine 10 is incorrectly configured or operated when used for “smaller patients”, who have a relatively small total blood volume compared to “regular patients”. As used herein, a smaller patient has a weight of 30 kg or less, and a regular patient has a weight in excess of 30 kg. In the context of the present disclosure, dialysis of a smaller patient is referred to as “pediatric dialysis”, even if the smaller patient is not an infant, child or adolescent. In pediatric dialysis, the rate of fluid removal (ultrafiltration rate, UFR) should be reduced compared to regular patients. Otherwise, there is a risk that the patient suffers from symptomatic hypotension, characterized by a blood pressure drop with symptoms in the form of cramps, nausea, vomiting and sometimes fainting. Such an event is not only strenuous for the patient, but also requires considerable attention from the staff overseeing the treatment. Thus, it is imperative that the caretaker enters appropriate control settings, via the UI device 14, when configuring the dialysis machine for pediatric dialysis. Dialysis machines are generally configured to perform a safety check of the control settings before initiating a therapy session, to ensure that the control settings are within safety limits and to alert the user if they are not. However, if the dialysis machine 10 is used for treatment of both regular patients and smaller patients, the safety limits are adapted to regular patients and the safety check may be unable to detect if the UFR is set too high for the smaller patient. This problem may apply to other control settings as well.

The technical solution capitalizes on the common practice to install a smaller pump segment 6′ in the blood pump 15 when pediatric dialysis is to be performed, to thereby reduce the blood flow rate generated per unit time by the blood pump 15. Conventionally, the caretaker enters the size of the installed pump segment 6′ into the dialysis machine 10, via the UI device 14, thereby allowing the dialysis machine 10 to operate the blood pump 15 to achieve a blood flow rate (BFR) in accordance with the control settings.

There is a general need to support a user that configures a dialysis machine for pediatric dialysis. In the context of the present disclosure, a “user” may be a caretaker, the patient, or any other individual that configures the dialysis machine for therapy.

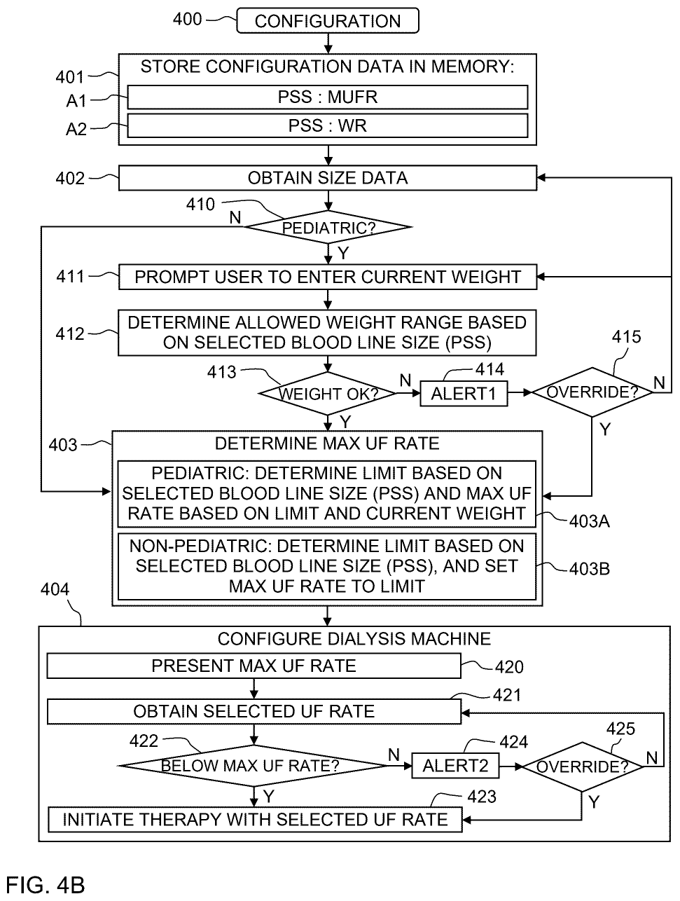

FIG. 4A is a flow chart of an example procedure 400 for configuring a dialysis machine for a session of extracorporeal blood therapy. The configuration procedure 400 will be described with reference to FIG. 3A. In step 401, configuration data (CD) is stored in memory 13′. As indicated in FIG. 4A, CD comprises an association A1 between blood line size (PSS) and a limit of fluid removal (MUFR). The limit of fluid removal represents a maximum value and may be given as an absolute UFR value (MUFRa) or as a weight-based UFR value (MUFRw). Thus, MUFRa may be seen to define a maximum UFR, and MUFRw may be seen to define a maximum UFR per unit weight of the patient. Given MUFRw, MUFRa may be determined as the product of MUFRw and the weight of the patient. Step 401 may be performed at any time in advance of a therapy session. For example, the configuration data may be predefined for all dialysis machines in a clinic and entered into the memory 13′ as a default setting for the dialysis machine 10. An example of the configuration data, CD, is shown in FIG. 3B. Here, PSS is represented by the inner diameter, D1-Dn (cf. FIGS. 2B-2C), and MUFR is given by a respective limit value LV1-LVn. In some embodiments, D1-Dn represent at least one pump segment adapted for pediatric dialysis and at least one pump segment adapted for use with regular patients.

In step 402, the control device 13 obtains size data indicative of a selected PSS for the upcoming therapy session. The selected PSS represents the pump segment 6′ that is or will be installed on the blood pump 15 for use in the upcoming therapy session. The size data may be given as an inner diameter, a serial number, or any other unique identifier. In some embodiments, the size data is entered by the user via the UI device 14. In other embodiments, the size data is inferred from an output signal of the reader 20 (FIG. 3A). The reader 20 may be arranged in association with the blood pump 15 to automatically read the identification code of the pump segment when pump segment is installed on the pump head (cf. FIG. 2A). Alternatively, control device 13 may prompt the user to bring the identification code within range of the reader 20 before or after installation.

In step 403, the control device 13 determines the maximum UF rate (MUFRa) by use of the configuration data and based on the selected PSS given by the size data. Thus, step 403 may comprise retrieving CD from memory 13′ and using the association A1 to determine MUFRa based on the selected PSS, either directly or by use of MUFRw.

In step 404, the control device 13 configures the dialysis machine 10 to maintain UFR below MUFRa throughout the therapy session. As noted above, the user may enter a control setting for UFR (“UFR setting”) via the UI device 14 before the therapy session. The UFR setting, also denoted “selected UFR” herein, may be a fixed value or a time profile of UFR. In some embodiments, step 404 comprises evaluating the UFR setting to ensure that it does not exceed MUFRa before initiating the therapy session, and requesting the user to change the UFR setting if deemed necessary.

The procedure 400 will provide user support to ensure that the dialysis machine 10 is not configured, in terms of UFR, to jeopardize the health of the patient irrespective of the size/weight/age of the patient. The procedure 400 enables the dialysis machine 10 to perform an automatic check of the UFR setting whenever a pediatric pump segment 6′ of known size is installed in the dialysis machine 10. Further, the provision of predefined configuration data enables simple updating of the predefined limit values LV1-LVn, for example in accordance with the needs of a specific clinic, ward or dialysis machine. Still further, the configuration data may be simply updated whenever a new PSS is made available for installation on the dialysis machine. The procedure 400 is simple to implement on pre-existing dialysis machines, by updating the control program stored in the control device 13, and optionally by installing the reader 20. The procedure 400 is also appliable to any available modality of extracorporeal blood therapy.

It is realized that if a MUFRw value is included in the configuration data, the weight of the patient needs to be known or estimated to enable calculation of a corresponding MUFRa value. In some embodiments, to simplify deployment, the configuration data may therefore be defined to only include MUFRa values, which are associated with PSS values. However, by including MUFRw values in the configuration data, a greater diversity of MUFRa values is achieved. The provision of MUFRw values may be particularly desirable for smaller patients, where the sensitivity to UFR may differ considerably with the total blood volume of the patient. In some embodiments, the configuration data associates pump segments adapted for smaller patients (“pediatric pump segments”) with a respective MUFRw value in the range of 8-10 ml/kg/h. Pediatric pump segments may have an inner diameter below 8 mm, such as 6.4 mm or 4 mm. It is conceivable that pediatric pump segments of different sizes are associated with the same MUFRw value. On the other hand, a pump segment adapted for regular patients (“regular pump segment”) may be associated with a MUFRa value, for example in the range of 1000-2000 ml/h, or a MUFRw value in the range of 12-14 ml/kg/h. Regular pump segments may have an inner diameter of 8 mm or larger.

FIG. 4B shows another example of the configuration procedure 400. In this example, the configuration data includes a MUFRw value, which is converted into MUFRa value by use of the current weight of the patient. Further, the configuration procedure 400 performs an additional check that the selected PSS is appropriate for the current weight of the patient. To enable the additional check, the configuration data comprises, in addition to the association A1, a further association A2 between blood line size (PSS) and an allowable weight range (WR). Reverting to the configuration data in FIG. 3B, the association A2 is represented by the provision of weight range values ΔW1-ΔWn.

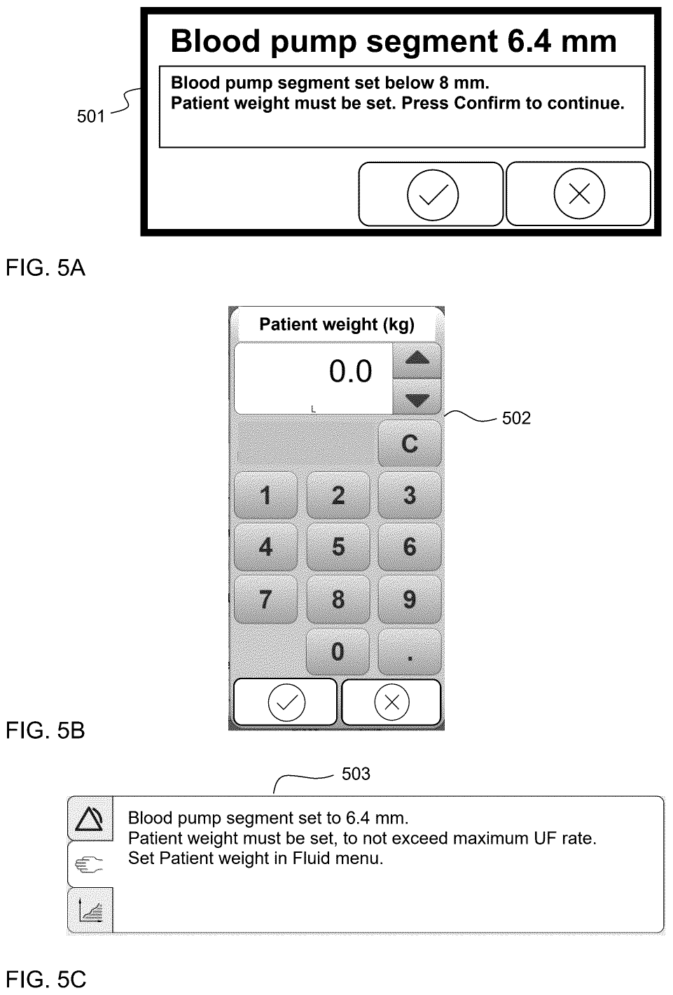

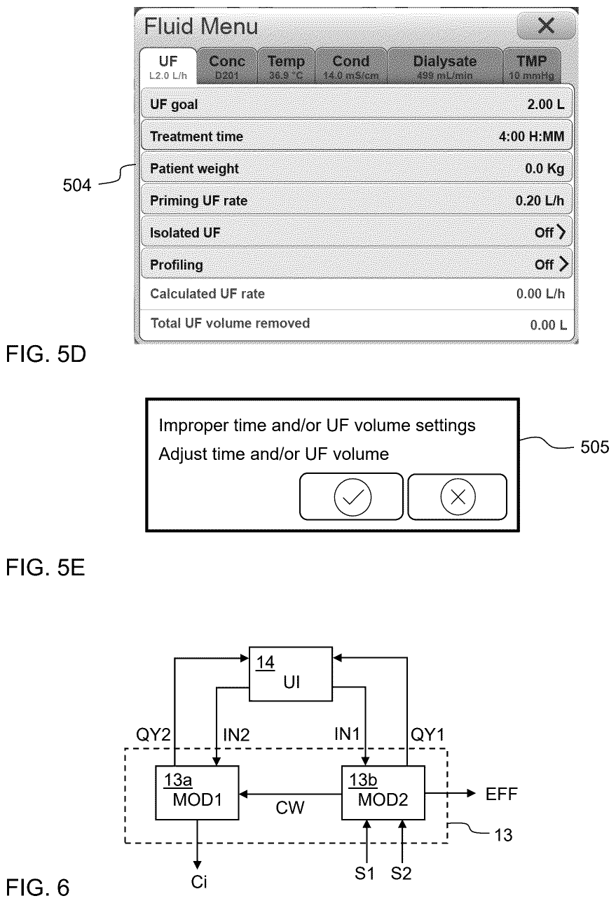

Like in FIG. 4A, the procedure 400 in FIG. 4B comprises a step 401 of storing the configuration data in memory 13′, and a step 402 of obtaining size data indicative of the selected PSS. The procedure 400 comprises intermediate steps 410-415 between step 402 and step 403. In step 410, the control device 13 checks if the selected PSS is associated with pediatric dialysis, for example by comparing the selected PSS to a predefined range of sizes used for pediatric dialysis. If not, the step 410 proceeds to step 403. Otherwise, step 410 proceeds to step 411, in which the user is prompted to enter the current weight of the patient, via the UI device 14. FIG. 5A shows an example of a dialog box 501 that may be presented on a screen of the UI device 14 in step 411. If the user activates the cancel button (x) in the dialog box 501, step 411 may return to step 402. If the user activates the confirm button (√), step 411 may present the interface 502 of FIG. 5B, which allows the user to manually enter the current weight of the patient and then activate the confirm button (√) to submit the current weight to the control device 13. The user may postpone entry of the current weight by activating the cancel button (x). If weight entry is postponed, the control device 13 may be configured to present a persistent message on the UI device 14 informing the user of the need to enter the current weight, for example message 503 in FIG. 5C. As shown, the message 503 directs the user to a dedicated menu (“Fluid menu”) for entering the current weight. FIG. 5D shows an example of such a menu 504, which is arranged to enable the user to enter control settings and view various properties of the dialysis machine or the therapy. In FIG. 5D, the user has activated the tab “UF”, in which the user is given the ability to enter UF-related control settings, including total UF (“UF goal”), treatment time, and current weight (“Patient weight”).

As used herein, “weight” is any parameter that correlates with the total body water in the patient. For example, weight may be given as body mass, body volume, body height, body water volume, body water mass, distribution volume, etc.

In a variation of step 411, the control device 13 obtains the current weight of the patient from an electronic medical record (EMR) or from a dedicated measurement device connected to the control device 13, for example a weighing scale.

In step 412, the control device 13 determines the allowed weight range for the selected PSS, by use of the configuration data. In the example of FIG. 3B, step 412 comprises accessing the configuration data, CD, and determining the WR value that is associated with the selected PSS. In step 413, the current weight is evaluated in relation to the weight range given by the WR value. If the current weight falls within this weight range, step 413 proceeds to step 403. Otherwise, step 413 proceeds to step 414, in which a weight alert (ALERT1) is presented on the UI device 14. The weight alert may indicate the allowable weight range and prompt the user to change the selected PSS. Thus, step 413 addresses the risk that the user installs a pediatric pump segment with an inappropriate size in the blood pump 15.

As indicated, step 415 may allow the user to override the request to change the selected PSS. Unless an override command is entered, step 415 proceeds to step 402. The override command causes step 415 to proceed to step 403.

Like in FIG. 4A, step 403 determines the maximum UF rate (MUFRa) by use of the configuration data and based on the selected PSS given by the size data. However, in the illustrated example, step 403 is performed differently depending on the outcome of step 410. If the selected PSS is associated with non-pediatric dialysis (regular patients), step 403B is performed. Step 403B does not account for the current weight of the patient. Instead, MUFRa is given by the limit value (LV1-LVn) that is associated with the selected PSS (D1-Dn) in the configuration data (cf. FIG. 3B). On the other hand, if the selected PSS is associated with pediatric dialysis, step 403A is performed. Step 403A accounts for the current weight of the patient, by obtaining MUFRw for the selected PSS and multiplying MUFRw and the current weight to generate MUFRa. It is realized that step 403A may involve an operation to convert the MUFRw and/or the current weight so that they are given in the same unit of weight, for example body mass, body water volume, etc.

Like in FIG. 4A, step 404 configures the dialysis machine for the upcoming therapy session. In the illustrated example, step 404 comprises sub-steps 420-425. In step 420, the control device 13 presents MUFRa to the user on the UI device 14. In step 421, the control device 13 obtains an UFR setting (“selected UFR”) for the upcoming therapy session, for example via the UI device 14. It is realized that the presentation in step 420 may assist the user in determining the UFR setting. In step 421, the UFR setting may be entered directly by the user or be calculated based on other control settings entered by the user. For example, in accordance with conventional practice, the user may enter total UF and treatment time, causing the control device 13 to calculate the UFR setting by diving total UF by treatment time. In step 422, the UFR setting is evaluated in relation to MUFRa. If the UFR setting is below MUFRa, step 422 proceeds to step 423, in which the control device 13 initiates the therapy session in accordance with the UFR setting and other control settings, which may be entered by the user or determined otherwise. If the UFR setting exceeds MUFRa, step 422 proceeds to step 424, in which an alert (ALERT2) is presented on the UI device 14. The alert may indicate that the UFR setting violates a dialysis protocol and prompt the user to change the UFR setting. As indicated, step 425 may allow the user to override the request to change the UFR setting. Unless an override command is entered, step 425 proceeds to step 421. The override command causes step 425 to proceed to step 423. FIG. 5E shows an example of a dialog box 505 that may be presented on a screen of the UI device 14 in step 424. If the user activates the confirm button (V) in the dialog box 505, step 424 proceeds to step 421. An override is performed if the user activates the cancel button (x).

In FIG. 4B, the user first identifies the selected PSS to the control device 13 (step 402), and optionally the current weight (step 411), and then inputs the UFR setting for the upcoming therapy session (421), whereupon the control device 13 is operated to validate the UFR setting (step 422) before initiating the therapy session (step 423). In a variant, the user first inputs the UFR setting (step 421), and then identifies the selected PSS to the control device 13 (step 402), and optionally the current weight (step 411), whereupon the control device 13 is operated to validate the UFR setting (step 422). In this variant, step 420 will be omitted.

In another variation of FIG. 4B, step 410 is omitted and intermediate steps 411- 415 are performed for any selected PSS. Further, step 403B may be performed to account for the current weight in correspondence with step 403A.

FIG. 6 is a block diagram of a control device 13 that comprises two separate program modules 13a, 13b, each containing program code (software) that is configured for execution by processor(s) in the control device 13 (cf. 133 in FIG. 1C). Program module 13a (MOD1) is configured to control the dialysis machine to perform a therapy session in accordance with conventional practice, by generating control signals (Ci) for functional components in the dialysis machine 10, such as the first and second pumping arrangements 15, 17 (FIG. 3A). MOD1 is also configured to perform the configuration procedure 400. Program module 13b (MOD2) is configured to evaluate the treatment efficiency (EFF) of the therapy session, based on output signals S1, S2 from the conductivity sensors 19a, 19b (FIG. 1B) and further based on the weight of the patient before the therapy session. For example, MOD2 may be configured in accordance with the DIASCAN® monitoring system from Baxter or the OCM® monitoring system from Fresenius. As shown, MOD2 is configured to present a query QY1 on the UI device 14, prompting the user to enter the current weight, which is included in input data IN1 for MOD2. When performing the configuration procedure 400, MOD1 presents one or more queries QY2 on the UI device 14, prompting the user to enter the size data (step 402) and the UFR setting (step 421), which form input data IN2 for MOD2. Further, as shown, MOD1 is configured to obtain the current weight (CW) by use of MOD2. For example, MOD1 may read CW from memory, if MOD2 has already received and stored CW in memory. Alternatively, MOD1 may prompt the user, via the UI device 14, to invoke a dedicated dialog box that is available through MOD2 and enter the current weight via the dedicated dialog box. In a further alternative, MOD1 may provide an instruction to MOD2 to invoke the dedicated dialog box, provided that MOD2 provides an appropriate interface (API) for receiving external instructions. By configuring MOD1 to obtain CW via MOD2, MOD1 is simplified and may be prepared by minimum modification of conventional program code for controlling a dialysis machine.

While the subject of the present disclosure has been described in connection with what is presently considered to be the most practical embodiments, it is to be understood that the subject of the present disclosure is not to be limited to the disclosed embodiments, but on the contrary, is intended to cover various modifications and equivalent arrangements included within the spirit and the scope of the appended claims.

Further, while operations are depicted in the drawings in a particular order, this should not be understood as requiring that such operations be performed in the particular order shown or in sequential order, or that all illustrated operations be performed, to achieve desirable results.

1. A dialysis machine, comprising:
a first pumping arrangement, which is configured to interface with a disposable blood line in fluid communication with a first compartment of a dialyzer, said the disposable blood line being available in at least two different sizes,
a second pumping arrangement which is configured to interface with a line set in fluid communication with a second compartment of the dialyzer, the second compartment being separated from the first compartment by a semipermeable membrane, the second pumping arrangement being operable to control fluid removal from blood in the first compartment through the semipermeable membrane,
a user interface for interaction with a user of the dialysis machine,
a memory for storing configuration data that associates the at least two different sizes of the disposable blood line with a respective predefined limit value of the fluid removal, and
a controller connected to the memory and the user interface and configured to operate the first and second pumping arrangements to perform extracorporeal blood therapy,
wherein the controller is further configured, during a configuration procedure, to:
obtain size data indicative of a selected size of the disposable blood line for use in the extracorporeal blood therapy in relation to a patient,
determine, by use of the configuration data and based on the selected size, a maximum rate of the fluid removal from blood, and
configure the dialysis machine to maintain the fluid removal below the maximum rate during the extracorporeal blood therapy.

2. The dialysis machine of claim 1, wherein the controller is configured to obtain the size data through the user interface.

3. The dialysis machine of claim 1, wherein the configuration data comprises at least one predefined limit value that is specific to pediatric dialysis and at least one predefined limit value that is specific to non-pediatric dialysis.

4. The dialysis machine of claim 1, wherein the controller is further configured to: obtain a current weight of the patient, wherein the controller is configured to determine the maximum rate based on the current weight of the patient and the predefined limit value that is associated with the selected size.

5. The dialysis machine of claim 4, wherein the predefined limit value defines a maximum rate of fluid removal per unit weight.

6. The dialysis machine of claim 4, wherein the controller is configured to obtain the current weight by prompting the user to enter the current weight through the user interface.

7. The dialysis machine of claim 4, wherein the controller is configured to obtain the current weight only if the selected size falls within a specific size range.

8. The dialysis machine of claim 7, wherein the specific size range represents blood lines used for pediatric dialysis.

9. The dialysis machine of claim 6, wherein the controller comprises a first module configured to perform the configuration procedure, and a second module configured to estimate treatment efficiency of the extracorporeal blood therapy based on conductivity data from a sensor arrangement in the dialysis machine, the second module being operative to allow input of the current weight through the user interface, wherein the first module is configured to obtain the current weight by use of the second module.

10. The dialysis machine of claim 4, wherein the configuration data further associates the different sizes with a respective weight range, and wherein the controller is further configured to evaluate the current weight in relation the weight range associated with the selected size of the disposable blood line and, if the current weight deviates from the weight range, present a weight alert on the user interface.

11. The dialysis machine of claim 1, wherein the controller is further configured to present the maximum rate on the user interface.

12. The dialysis machine of claim 1, wherein the controller is further configured to determine, based on input data obtained through the user interface, a selected rate of fluid removal for use in the extracorporeal blood therapy, evaluate the selected rate in relation to the maximum rate, and, if the selected rate exceeds the maximum rate, present a rate alert on the user interface.

13. The dialysis machine of claim 1, wherein the first pumping arrangement comprises a peristaltic pump, and wherein the at least two different sizes correspond to different inner diameters of a portion of the disposable blood line configured for engagement with one or more rollers of the peristaltic pump.

14. A computer-implemented method of configuring a dialysis machine to perform extracorporeal blood therapy, the dialysis machine comprising a user interface; a first pumping arrangement, which is configured to interface with a disposable blood line in fluid communication with a first compartment of a dialyzer, the disposable blood line being available in at least two different sizes; and a second pumping arrangement which is configured to interface with a line set in fluid communication with a second compartment of the dialyzer, the second compartment being separated from the first compartment by a semipermeable membrane, the second pumping arrangement being operable to control fluid removal from blood in the first compartment through the semipermeable membrane during the extracorporeal blood therapy, the computer-implemented method comprising:
obtaining size data indicative of a selected size of the disposable blood line for use in the extracorporeal blood therapy in relation to a patient;
retrieving, from a memory, configuration data that associates the at least two different sizes of the disposable blood line with a respective predefined limit value of the fluid removal;
determining, by use of the configuration data and based on the selected size, a maximum rate of said fluid removal from blood; and
configuring the dialysis machine to maintain the fluid removal below the maximum rate during the extracorporeal blood therapy.

15. A computer-readable medium comprising computer instructions, which, when executed by a processor, cause the processor to perform the method of claim 14.
